(19) United States
(12) Patent Application Publication
(10) Pub. No.: US 2010/0215722 A1
(43) Pub. Date: Aug. 26, 2010

(54) Tissue Regeneration II

(76) Inventors: Vishal Bhasin, New South Wales (AU); Helder Marcal, New South Wales (AU)

Correspondence Address: MEDLEN & CARROLL, LLP, 101 HOWARD STREET, SUITE 350, SAN FRANCISCO, CA, 94105, US

(21) Appl. No.: 12/224,470

(22) PCT Filed: Feb. 28, 2007
(86) PCT No.: PCT/AU2007/000238

§ 371 Date: Nov. 17, 2009

(30) Foreign Application Priority Data
Feb. 28, 2006 (NZ) 545598
Sep. 29, 2006 (NZ) 550221

(51) Int. Cl. A61K 9/70; A61K 38/18; A61K 35/12; A61K 9/00; A61P 25/00

(52) U.S. Cl. 424/445; 514/12; 424/93.7; 424/400

(57) ABSTRACT

The invention relates to tissue regeneration and to therapeutic compositions, formulations and dressings.

### FIELD OF THE INVENTION

The invention relates to tissue regeneration and to therapeutic compositions, formulations and dressings.

### BACKGROUND OF THE INVENTION

Tissue regeneration is a process that typically involves the remodelling and/or replacement of tissue elements, such as cellular and extracellular elements. For many invertebrate species, the process is important for asexual reproduction. Further, for these and some vertebrate species, the process is essential for maintenance of tissue structure and function and for restoration of tissue structure and function after tissue injury. Typically, a hallmark of tissue regeneration is the creation of new tissue that has structure and function that is comparable with old tissue.

Tissue regeneration is distinguished from wound healing. The latter occurs in response to tissue injury only and typically involves angiogenesis and fibrosis, resulting in granulation and the formation of scar tissue. A key difference between tissue regeneration and wound healing is that the scar tissue formed from wound healing of a tissue does not have structure and function that is comparable to the tissue prior to wounding.

Tissue regeneration, in the form of vasculogenesis is also different from angiogenesis. A hallmark of angiogenesis, whether associated with normal or abnormal physiology, is the formation of new blood vessels from an existing vascular bed. In normal physiology, angiogenesis is observed during embryonic development, during the female reproductive cycle and, as noted above, during wound repair. In these circumstances, angiogenesis is tightly regulated and is limited by the metabolic demands of the tissues concerned. Angiogenesis is also observed in a number of pathologies, including tumorigenesis, inflammation and various autoimmune conditions. In these circumstances, regulation of angiogenesis appears to be lost.

In comparison, tissue regeneration in the form of vasculogenesis is a series of differentiation and morphogenetic events which result in the formation of a primary capillary plexus. The process typically has three stages; 1) the in situ differentiation of mesodermal cells into angioblasts, 2) the differentiation of angioblasts into endothelial cells, 3) the organisation of newly formed endothelial cells into a primary plexus. The primary differentiation of mesodermal angioblasts is a process that is limited exclusively to vasculogenesis. It does not occur during angiogenesis because mesoderm does not persist into post-natal life. By definition vasculogenesis must precede angiogenesis, although the two processes continue in parallel during early development. Unlike vasculogenesis which appears to be restricted to early development, angiogenesis is also required for the maintenance of functional and structural integrity of the organism in post-natal life.

As noted above, tissue regeneration is an essential physiological process for many invertebrate species and some vertebrates. In some invertebrate species, such as planarians, existing stem cells, known as neoblasts, are essential for regenerating tissue. Such a regenerative capacity depends on the activation, proliferation, and differentiation of the neoblasts.

The axolotl is one of the few vertebrate species in which tissue regeneration has been observed during adult life. Evidence suggests that these amphibians regenerate amputated and/or injured structures such as their limbs, tails and eyes through a process that involves the formation of a blastema and subsequent activation of a regenerative process reminiscent of the developmental program that originally functioned to specify the original respective structure. It is believed that the blastema consists of progenitor cells that form from the dedifferentiation of terminally differentiated cells existing at a site of amputation. However, it is equally possible that multipotent adult stem cells exist in adult axolotl tissue, and indeed at a wound site, and that these cells, rather than dedifferentiated mature cells, are the progenitor cells that are required for tissue regeneration.

Post-natal mammalian tissue, and especially adult tissues seem to be incapable of tissue regeneration. Dedifferentiation of terminally differentiated cells in adult mammals has not been clearly and unequivocally documented, and at present, no evidence directly supports transdifferentiation or dedifferentiation events as an explanation for stem cell plasticity in vivo.

Approaches for potentiating the regenerative capacity of mammalian tissue are: (1) transplantation of stem or progenitor cells, or differentiated cells into a compromised tissue or organ; (2) transplantation of cell-seeded scaffolds (biodegradable, biocompatible, bio-mimetic) into damaged regions; and (3) induction of endogenous regeneration through exogenous addition of compounds to activate stem cells or mobilise a pool of stem cells from the surrounding tissue and expand them through stimulation of cellular proliferation and chemotaxis.

### SUMMARY

In certain embodiments there is provided a method for inducing regeneration of a mammalian tissue at a site of injury in the tissue, the method including the steps of:

providing an agent selected from the group consisting of:

a neuregulin;

a fragment of a neuregulin;

a compound for inducing expression of a neuregulin gene;

an agonist or antagonist of a receptor for neuregulin;

to a site of injury in a mammalian tissue to induce regeneration of the tissue at the site of injury.

In other embodiments there is provided a composition for inducing regeneration of a mammalian tissue at a site of injury in the tissue, the composition including at least one of:

a neuregulin;

a fragment of a neuregulin;

a compound for inducing expression of a neuregulin gene; and

an agonist or antagonist of a receptor for neuregulin.

In related embodiments the composition includes a neurotrophin, especially nerve growth factor (NGF).

In other embodiments there is provided a use of a composition described above in the manufacture of a medicament for inducing regeneration of a mammalian tissue at a site of injury in the tissue.

In other embodiments there is provided a dressing for inducing tissue regeneration of a mammalian tissue at a site of injury, the dressing including a composition as described above.

In other embodiments there is provided a method for inducing regeneration of a mammalian tissue at a site of injury in a tissue, the method including the steps of:

providing an agent selected from the group consisting of:

a neurotrophin, such as nerve growth factor (NGF), neurotrophic factor 3 (NT-3), brain derived neurotrophic factor (BDNF), neurotrophic factor 4 (NT-4), neurotrophic factor 5 (NT-5) and Ciliary Neurotrophic Factor CNTF;

a fragment of a neurotrophin;

a compound for inducing expression of a neurotrophin gene;

an agonist or antagonist of a receptor for a neurotrophin to a site of injury in a mammalian tissue to induce regeneration of the tissue at the site of injury.

In further embodiments there is provided a composition for inducing regeneration of a mammalian tissue at a site of injury in the tissue, the composition including:

a neurotrophin, such as nerve growth factor (NGF), neurotrophic factor 3 (NT-3) brain derived neurotrophic factor (BDNF), neurotrophic factor 4 (NT-4), neurotrophic factor 5 (NT-5) and Ciliary Neurotrophic Factor (CNTF);

a fragment of a neurotrophin,

a compound for inducing expression of a neurotrophin gene;

an agonist or antagonist of a receptor for a neurotrophin.

In related embodiments, the composition includes a neuregulin.

In other embodiments there is provided a use of a composition as discussed above in the manufacture of a medicament for inducing regeneration of a mammalian tissue at a site of injury in the tissue.

In other embodiments there is provided a dressing for inducing tissue regeneration of a mammalian tissue at a site of injury, the dressing including a composition as discussed.

### DETAILED DESCRIPTION OF THE EMBODIMENTS

The inventors have found that neuregulin (otherwise known as “neu differentiation factor (NDF)”, “neu”, “HER-2 ligand”, “c-erb-2 ligand”) and certain neurotrophins, especially nerve growth factor (NGF) (also known as “p75NTR ligand”, “CD271 ligand” and “low affinity nerve growth factor receptor (LNGFR) ligand”), and neutrotrophic factor 3 (also known as “NT-3”) are associated with regeneration of certain tissue, especially mammalian tissue.

While not wanting to be bound by hypothesis, the inventors note that neuregulin, NGF and NT-3 may have roles in the induction of plasticity in determined cells, in the recruitment of multipotent satellite cells and/or in the proliferation of these cells and hence the production of differentiated cells capable of restoring injured tissue to a structural and functional phenotype of the tissue before injury.

It will be understood that improvements in tissue regeneration may be obtained using neuregulin or a neurotrophin such as NGF or NT-3, or typically a combination of at least two of these molecules.

In certain embodiments there is provided a composition for inducing regeneration of a mammalian tissue at a site of injury in the tissue. The composition includes one or more of: a neuregulin; a fragment of a neuregulin; a compound for inducing expression of a neuregulin gene; and an agonist or antagonist of a neuregulin receptor.

Generally speaking, neuregulin is otherwise known as “NDF”, “neu”, “HER-2 ligand”, or “c-erb-2 ligand. More broadly a neuregulin is a molecule that is secreted from a cell and that is capable of directly or indirectly causing tyrosine phosphorylation of a Her-2 (c-erb-2) domain.

Other examples of neuregulin include heregulin, sensory and motor neuron-derived factor (SMDF), acetylcholine receptor inducing activity (ARIA), glial growth factor (GGF), HGL, HRGA, breast cancer cell differentiation factor 45.

The neuregulin for use in the composition is typically syngeneic. In other words, if the mammalian tissue is human tissue, the neuregulin is typically human neuregulin, otherwise known as heregulin. However, in some circumstances improved tissue regeneration is observed with other neuregulin molecules.

In other circumstances, certain fragments of a neuregulin, such as a naturally occurring isoform may provide improved tissue regeneration. Examples of naturally occurring isoforms include pro-NDF β3, pro-NDF α2b, pro-NDF β2a, pro-NDF α2a truncated, pro-NDF β2, pro-NDF β4α, pro-NDF α2c and pro-NDF α2a. See: Wen et al. 1994 Mol. Cell Biol. 14: 1909-1919.

An example of neuregulin includes a peptide having an amino acid sequence shown in SwissProt accession number Q02297 or O14511.

It will be understood, however, that the neuregulin may have an amino acid sequence that is not the same as, but rather, has homology with the sequences referenced by SwissProt references Q02297. These are described herein as a “neuregulin variant”. In some embodiments, an neuregulin variant has a sequence that is at least 70% homologous to the sequence referenced by SwissProt reference Q02297 shown above. In other embodiments the neuregulin variant sequence is at least 75% homologous to the sequence referenced by SwissProt reference Q02297 shown above. In other embodiments the neuregulin variant sequence is at least 80% homologous to the sequence referenced by SwissProt reference Q02297 shown above. In other embodiments the neuregulin variant sequence is at least 85% homologous to the sequence referenced by SwissProt reference Q02297 shown above. In other embodiments the neuregulin variant sequence is at least 90% homologous to the sequence referenced by SwissProt reference Q02297 shown above. In other embodiments the neuregulin variant sequence is at least 95% homologous to the sequence referenced by SwissProt reference Q02297 shown above. In other embodiments the neuregulin variant sequence is at least 98% homologous to the sequence shown in SEQ ID No: 1. In other embodiments the neuregulin variant sequence is at least 99% homologous to the sequence referenced by SwissProt reference Q02297 shown above.

It will be understood that in determining homology, regard is not to be had to peptide regions that are not included in a peptide that has been subjected to post translational modifications, such as, for example a leader or signal peptide.

As described herein, neuregulin, including human and non human neuregulin, fragments thereof and neuregulin variants, may be prepared by chemical synthesis methodologies or by recombinant DNA technology. For example, these agents can be prepared from monomers using a chemical synthesis methodology based on the sequential addition of amino acid residues, for example as described in Merrifield, J. Am. Chem. Soc., 85: 2149 (1963). These monomers may be naturally occurring residues, or non naturally occurring residues, examples of which are described below. Alternatively, the agents, and in particular, a neuregulin fragment, can be prepared by enzymatically or chemically treating a peptide having, for example, a sequence referenced by SwissProt reference Q02297 or O14511 shown above. Where these peptides are to be synthesised by recombinant DNA technology, they may be prepared by random or pre-determined mutation (eg site directed PCR mutagenesis) of a nucleic acid molecule that encodes a neuregulin sequence, for example, a sequence referenced by SwissProt reference Q02297 or O14511 and expression of the sequence in a host cell to obtain the peptide. This is a particularly useful process for preparing neuregulin variants. An alternative process is de novo chemical synthesis of a nucleic acid molecule that encodes a sequence referenced by SwissProt reference Q02297 or O14511 shown above, or a sequence that is homologous to the sequence referenced by SwissProt reference Q02297 or O14511 shown above and expression of the sequence in the host cell to obtain the peptide.

The peptides that are variants of the sequence referenced by SwissProt reference Q02297 or O14511 shown above typically differ in terms of one or more conservative amino acid substitutions in these sequences. Examples of conservative substitutions are shown in Table 1 below.

TABLE 1

| | Exemplary | Preferred |
|---|---|---|
| Original | Conservative | Conservative |
| Residue | Substitution | Substitution |
| Ala | Val, Leu, Ile | Val |
| Asn | Gln Lys His Phe | Gln |
| Gln | Asn | Asn |
| Gly | Pro | Pro |
| Ile | Leu, Val, Met, | Leu |
| | Ala, Phe | |
| Leu | Ile, Val, Met, | Ile |
| | Ala, Phe | |
| Lys | Arg, Gln, Asn | Arg |
| Phe | Lue, Val, | Leu |
| | Ile, Ala | |
| Pro | Gly | Gly |
| Ser | Thr | Thr |
| Val | Ile, Leu, Met, | Leu |
| | Phe, Ala | |
| Asp | Glu | Glu |
| Thr | Ser | Ser |
| Trp | Tyr | Tyr |
| Tyr | Trp Phe Thr Ser | Phe |

As noted above, the human and non human neuregulin and fragments thereot, and neuregulin variants, may include non naturally occurring amino acid residues. Commonly encountered amino acids which are not encoded by the genetic code, include:

2-amino adipic acid (Aad) for Glu and Asp;

2-aminopimelic acid (Apm) for Glu and Asp;

2-aminobutyric (Abu) acid for Met, Leu, and other aliphatic amino acids;

2-aminoheptanoic acid (Ahe) for Met, Leu and other aliphatic amino acids;

2-aminoisobutyric acid (Aib) for Gly;

cyclohexylalanine (Cha) for Val, and Leu and Ile;

homoarginine (Har) for Arg and Lys;

2,3-diaminopropionic acid (Dpr) for Lys, Arg and His;

N-ethylglycine (EtGly) for Gly, Pro, and Ala;

N-ethylasparigine (EtAsn) for Asn, and Gln;

Hydroxyllysine (Hyl) for Lys;

allohydroxyllysine (AHyl) for Lys;

3-(and 4) hydroxyproline (3Hyp, 4Hyp) for Pro, Ser, and Thr;

alloisoleucine (Alle) for Ile, Leu, and Val;

ρ-amidinophenylalanine for Ala;

N-methyigiycine (MeGly, sarcosine) for Gly, Pro, Ala.

N-methylisoleucine (Melle) for Ile;

Norvaline (Nva) for Met and other aliphatic amino acids;

Norleucine (Nle) for Met and other aliphatic amino acids;

Ornithine (Orn) for Lys, Arg and His;

Citrulline (Cit) and methionine sulfoxide (MSO) for Thr, Asn and Gln;

N-methylphenylalanine (MePhe), trimethyiphenylalanine, halo (F, Cl, Br and I) phenylalanine, triflourylphenylalanine, for Phe.

A useful method for identification of a residue of the sequence referenced by SwissProt reference Q02297 or O14511 shown above for amino acid substitution to generate a neuregulin variant is called alanine scanning mutagenesis as described by Cunningham and Wells (1989) Science, 244:1081-1085. Here a residue or group of target residues are identified (eg charged residues such as Asn, Gln and Lys) and replaced by a neutral or negatively charged amino acid to affect the interaction of the amino acids with the surrounding environment. Those domains demonstrating functional sensitivity to the substitution then are refined by introducing further or other variations at or for the sites of substitution. Thus while the site for introducing an amino acid sequence variation is predetermined the nature of the mutation per se need not be predetermined. For example, to optimize the performance of a mutation at a given site, Ala scanning or random mutagenesis may be conducted at the target codon or region and the expressed peptide screened for the optimal combination of desired activity.

Phage display of protein or peptide libraries offers another methodology for the selection of peptide with improved or altered affinity, specificity, or stability (Smith, G, P, (1991) Curr Opin Biotechnol (2:668-673). High affinity proteins, displayed in a monovalent fashion as fusions with the M13 gene III coat protein (Clackson, T, (1994) et al, Trends Biotechnol 12:173-183), can be identified by cloning and sequencing the corresponding DNA packaged in the phagemid particles after a number of rounds of binding selection.

Human and non human neuregulin, fragments thereof and neuregulin variants may be prepared as the free acid or base or converted to salts of various inorganic and organic acids and bases. Examples of such salts include ammonium, metal salts like sodium, potassium, calcium and magnesium; salts with organic bases like dicyclohexylamine, N-methyl-D-glucamine and the like; and salts with amino acids like arginine or lysine. Salts with inorganic and organic acids may be likewise prepared, for example, using hydrochloric, hydrobromic, sulfuric, phosphoric, trifluoroacetic, methanesulfonic, malic, maleic, fumaric and the like. Non-toxic and physiologically compatible salts are particularly useful, although other less desirable salts may have use in the processes of isolation and purification.

In one embodiment, human or non human neuregulin, fragments thereof or neuregulin variants include at least one carbohydrate molecule and/or at least one lipid Molecule.

In one embodiment human or non human neuregulin, fragments thereof or neuregulin variants include a N-terminal fatty acid moiety, for example a C14 to C17 fatty acid of n, iso or antieso form.

In one embodiment, human or non human neuregulin, fragments thereof or neuregulin variants include at least one akyl group.

The human or non human neuregulin, fragments thereof or neuregulin variants may include a further peptide, for example for controlling degradation of the peptide, or for arranging the peptide on a solid phase or for binding with an antibody or receptor to purify, isolate or detect the peptide. Such peptides are otherwise known in the art as fusion proteins.

Fusion proteins can be made by the chemical synthesis methods describe below, or they can be made by recombinant DNA techniques, for example, wherein a nucleic acid molecule encoding the peptide having the sequence referenced by SwissProt reference Q02297 or O14511 shown above is arranged in a vector with a gene encoding another protein or a fragment of another protein. Expression of the vector results in the human or non human neuregulin, fragments thereof or neuregulin variants being produced as a fusion with another protein or peptide.

The further protein or peptide that is fused to the human or non human neuregulin, fragments thereof or neuregulin variants may be a protein or peptide that can be secreted by a cell, making it possible to isolate and purify from the culture medium and eliminating the necessity of destroying the host cells; this necessity arises when human or non human neuregulin, fragments thereof or neuregulin variants remains inside the cell. Alternatively, the fusion protein can be expressed inside the cell as a function of the further protein or peptide. It is useful to use fusion proteins that are highly expressed.

The use of fusion proteins, though not essential, can facilitate the expression of heterologous peptides in E. coli as well as the subsequent purification of those gene products. Harris, in Genetic Engineering, Williamson, R., Ed. (Academic Press, London, Vol. 4, 1983), p. 127; Liungquist et al., Eur. J. Biochem., 186: 557-561 (1989) and Liungquist et al., Eur. J. Biochem., 186: 563-569 (1989). Protein A fusions are often used because the binding of protein A, or more specifically the Z domain of protein A, to IgG provides an “affinity handle” for the purification of the fused protein. It has also been shown that many heterologous proteins are degraded when expressed directly in E. coli, but are stable when expressed as fusion proteins. Marston, Biochem J., 240: 1 (1986).

Fusion proteins can be cleaved using chemicals, such as cyanogen bromide, which cleaves at a methionine, or hydroxylamine, which cleaves between an Asn and Gly residue. Using standard recombinant DNA methodology, the nucleotide base pairs encoding these amino acids may be inserted just prior to the 5′ end of the gene encoding the desired peptide.

Alternatively, one can employ proteolytic cleavage of fusion protein, see for example Carter in Protein Purification: From Molecular mechanisms to Large-Scale Processes, Ladisch et al., eds. (American Chemical Society Symposium Series No. 427, 1990), Ch 13, pages 181-193.

Proteases such as Factor Xa, thrombin, and subtilisin or its mutants, and a number of others have been successfully used to cleave fusion proteins. Typically, a peptide linker that is amenable to cleavage by the protease used is inserted between the further proteins (e.g., the Z domain of protein A) and human or non human neuregulin, fragments thereof or neuregulin variants. Using recombinant DNA methodology, the nucleotide base pairs encoding the linker are inserted between the genes or gene fragments coding for the other proteins. Proteolytic cleavage of the partially purified fusion protein containing the correct linker can then be carried out on either the native fusion protein, or the reduced or denatured fusion protein.

The human or non human neuregulin, fragments thereof or neuregulin variants may not be properly folded when expressed as a fusion protein. Also, the specific peptide linker containing the cleavage site may or may not be accessible to the protease. These factors determine whether the fusion protein must be denatured and refolded, and if so, whether these procedures are employed before or after cleavage.

When denaturing and refolding are needed, typically human or non human neuregulin, fragments thereof or neuregulin variants is treated with a chaotrope, such as guanidine HCl, and is then treated with a redox buffer, containing, for example, reduced and oxidized dithiothereitol or glutathione at the appropriate ratios, pH, and temperature, such that the relevant peptide is refolded to its native structure.

Other fusion proteins include those wherein the human or non human neuregulin, fragments thereof or neuregulin variants is fused to a protein having a long half-life such as immunoglobulin constant region or other immunoglobulin regions, albumin, or ferritin.

The human or non human neuregulin, fragments thereof or neuregulin variants may be stabilized by polymerization. This may be accomplished by cross linking the human or non human neuregulin, fragments thereof or neuregulin variants with polyfunctional cross linking agents, either directly or indirectly, through multi-functional polymers. For example, two substantially identical polypeptides may be cross linked at their C- or N-termini using a bifunctional cross linking agent. The agent may be used to cross link the terminal amino and/or carboxyl groups. While both terminal carboxyl groups or both terminal amino groups may be cross linked to one another, other cross linking agents permit the alpha amino of one peptide to be cross linked to the terminal carboxyl group of the other peptide.

To facilitate use of other reagents for cross linking, the human or non human neuregulin, fragments thereof or neuregulin variants may be substituted at their C-termini with cysteine. Under conditions well known in the art a disulfide bond can be formed between the terminal cysteines, thereby cross linking peptide chains. For example, disulfide bridges are conveniently formed by metal-catalyzed oxidation of the free cysteines or by nucleophilic substitution of a suitably modified cysteine residue.

Selection of the cross linking agent will depend upon the identities of the reactive side chains of the amino acids present in the peptides. For example, disulfide cross linking would not be preferred if cysteine was present in the peptide at additional sides other than the C-terminus.

A further approach for cross linking peptides is the use of methylene bridges.

Suitable cross linking sites on the peptides, aside from the N-terminal amino and C-terminal carboxyl groups, include epsilon amino groups found on lysine residues, as well as amino, imino, carboxyl, sulfhydryl and hydroxyl groups located on the side chains of internal residues of the peptides or residues introduced into flanking sequences. Cross linking through externally added cross linking agents is suitably achieved, e.g., using any of a number of reagents familiar to those skilled in the art, for example, via carbodiimide treatment of the peptide. Other examples of suitable multi-functional (ordinarily bifunctional) cross linking agents are found in the literature.

The human or non human neuregulin, fragments thereof or neuregulin variants also may be conformationally stabilized by cyclization. They may be cyclized by covalently bonding the N- and the C-terminal domains of one peptide to the corresponding domain of another human or non human neuregulin, fragments thereof or neuregulin variants, so as to form cyclo-oligomers containing two or more iterated peptide sequences. Further, cyclized peptides (whether cyclo-oligomers or cyclo-monomers) may be cross linked to form 1-3 cyclic structures having from 2 to 6 peptides comprised therein. The peptides typically are not covalently bonded through α-amino and main chain carboxyl groups (head to tail), but rather are crosslinked through the side chains of residues located in the N- and C-terminal domains. The linking sites thus generally will be between the side chains of the residues.

Many suitable methods per se are known for preparing mono- or poly-cyclized peptides as contemplated herein. Lys/Asp cyclization has been accomplished using Na-Boc-amino acids on solid-phase support with Fmoc/9-fluorenylmethyl (OFm) side-chain protection for Lys/Asp; the process is completed by piperidine treatment followed by cyclization.

Glu and Lys side chains also have been crosslinked in preparing cyclic or bicyclic peptides: the peptide is synthesized by solid phase chemistry on a p-methylbenzhydrylamine resin. The peptide is cleaved from the resin and deprotected. The cyclic peptide is formed using disphenylphosphrylazide in diluted methylformamide. For an alternative procedure, see Schiller et al., Peptide Protein Res., 25: 171-177 (1985). See also U.S. Pat. No. 4,547,489.

Disulfide cross linked or cyclized peptides may be generated by conventional methods. The method of Pelton et al. (J. Med. Chem., 29: 2370-2375 (1986) is suitable. The same chemistry is useful for synthesis of dimers or cyclo-oliogomers or cyclo-monomers. Also useful are thiomethylene bridges. Lebl and Hruby, Tetrahedron Letters, 25: 2067-2068 (1984). See also Cody et al., J. Med. Chem., 28: 583 (1985).

The desired cyclic or polymeric peptides may be purified by gel filtration followed by reversed-phase high pressure liquid chromatography or other conventional procedures. The peptides may be sterile filtered for formulation into a therapeutic composition described further herein.

The human or non human neuregulin, fragments thereof or neuregulin variants described above can be made by chemical synthesis or by employing recombinant DNA technology. These methods are known in the art. Chemical synthesis, especially solid phase synthesis, is preferred for short (e.g., less than 50 residues) peptides or those containing unnatural or unusual amino acids such as D-Tyr, Ornithine, amino adipic acid, and the like. Recombinant procedures are preferred for longer peptides. When recombinant procedures are selected, a synthetic gene may be constructed de novo or a natural gene may be mutated by, for example, cassette mutagenesis. These procedures are described further herein. Set forth below are exemplary general procedures for chemical synthesis of human or non human neuregulin, fragments thereof or neuregulin variants.

Peptides are typically prepared using solid-phase synthesis, such as that generally described by Merrifield, J. Am. Chem. Soc., 85: 2149 (1963), although other equivalent chemical syntheses known in the art are employable. Solid-phase synthesis is initiated from the C-terminus of the peptide by coupling a protected α-amino acid to a suitable resin. Such a starting material can be prepared by attaching a α-amino-protected amino acid by an ester linkage to a chloromethylated resin or a hydroxymethyl resin, or by an amide bond to a BHA resin or MBHA resin. The preparation of the hydroxymethyl resin is described by Bodansky et al., Chem. Ind. (London), 38: 1597-1598 (1966). Chloromethylated resins are commercially available from BioRad Laboratories, Richmond, Calif. And from Lab. Systems, Inc. The preparation of such a resin is described by Stewart et al., “Solid Phase Peptide Synthesis” (Freeman & Co., San Francisco 1969), Chapter 1, pp. 1-6. BHA and MBHA resin supports are commercially available and are generally used only when the desired polypeptide being synthesized has an unsubstituted amide at the C-terminus.

The amino acids are coupled to the peptide chain using techniques well known in the art for the formation of peptide bonds. One method involves converting the amino acid to a derivative that will render the carboxyl group more susceptible to reaction with the free N-terminal amino group of the peptide fragment. For example, the amino acid can be converted to a mixed anhydride by reaction of a protected amino acid with ethychloroformate, phenyl chloroformate, sec-butyl chloroformate, isobutyl chloroformate, pivaloyl chloride or like acid chlorides. Alternatively, the amino acid can be converted to an active ester such as a 2,4,5-trichlorophenyl ester, a pentachlorophenyl ester, a pentafluorophenyl ester, a p-nitrophenyl ester, a N-hydroxysuccinimide ester, or an ester formed from 1-hydroxybenzotriazole.

Another coupling method involves use of a suitable coupling agent such as N,N¹-dicyclohexylcarbodiimide or N,N¹-diisopropylcarbodiimide. Other appropriate coupling agents, apparent in those skilled in the art, are disclosed in E Gross & J Meienhofer, The Peptides: Analysis, Structure, Biology, Vol. I: Major Methods of Peptide Bond Formation (Academic Press, New York, 1979).

It should be recognized that the α-amino group of each amino acid employed in the peptide synthesis must be protected during the coupling reaction to prevent side reactions involving their active α-amino function. It should also be recognized that certain amino acids contain reactive side-chain functional groups (eg sulfhydryl, amino, carboxyl, and hydroxyl) and that such functional groups must also be protected with suitable protecting groups to prevent a chemical reaction from occurring at that site during both the initial and subsequent coupling steps. Suitable protecting groups, known in the art, are described in Gross and Meienhofer, The Peptides: Analysis, Structure, Biology, Vol, 3: “Protection of Functional Groups in Peptide Synthesis” (Academic Press, New York 1981).

In the selection of a particular side-chain protecting group to be used in synthesizing the peptides, the following general rules are followed. An α-amino protecting group must render the α-amino function inert under the conditions employed in the coupling reacting, must be readily removable after the coupling reaction under conditions that will not remove side-chain protecting groups and will not alter the structure of the peptide fragment, and must eliminate the possibility of racemization upon activation immediately prior to coupling. A side-chain protecting group must render the side chain functional group inert under the conditions employed in the coupling reaction, must be stable under the conditions employed in removing the α-amino protecting group, and must be readily removable upon completion of the desired amino acid peptide under reaction conditions that will not alter the structure of the peptide chain.

It will be apparent to those skilled in the art that the protecting groups known to be useful for peptide synthesis will vary in reactivity with the agents employed for their removal. For example, certain protecting groups such as triphenylmethyl and 2-(p-biphenylyl)isopropyloxycarbonyl are very labile and can be cleaved under mild acid conditions. Other protecting groups, such as t-butyloxycarbonyl (BOC), t-amyloxycarbonyl, adamantyloxycarbonyl, and p-methoxybenzyloxycarbonyl are less labile and require moderately strong acid, such as trifluoroacetic, hydrochloric, or boron trifluoride in acetic acid, for their removal. Still other protecting groups, such as benzyloxy-carbonyl (CBZ or Z), halobenzyloxycarbonyl, p-nitrobenzyloxycarbonyl cycloalkyloxycarbonyl, and isopropyloxycarbonyl, are even less labile and require stronger acids, such as hydrogen fluoride, hydrogen bromide, or boron trifluoroacetate in trifluoroacetic acid, for their removal. Among the classes of useful amino acid protecting groups are included:

(1) for an α-amino group, (a) aromatic urethane-type protecting groups, such as fluorenylmethyloxycarbonyl (FMOC) CBZ, and substituted CBZ, such as, eg, p-chlorobenzyloxycarbonyl, p-6-nitrobenzyloxycarbonyl, p-bromobenzyloxycarbonyl, and p-methoxybenzyloxycarbonyl, o-chlorobenzyloxycarbonyl, 2,4-dichlorobenzyloxycarbonyl, 2,6-dichlorobenzyloxycarbonyl, and the like; (b) aliphatic urethane-type protecting groups, such as BOC, t-amyloxycarbonyl, isopropyloxycarbonyl, 2-(p-biphenylyl)-isopropyloxycarbonyl, allyloxycarbonyl and the like; (c) cycloalkyl urethane-type protecting groups, such as cyclopentyloxycarbonyl, adamantyloxycarbonyl, and cyclohexyloxycarbonyl; and (d) allyloxycarbonyl. The preferred α-amino protecting groups are BOX or FMOC.

(2) for the side chain amino group present in Lys, protection may be by any of the groups mentioned above in (1) such as BOC, p-chlorobenzyloxycarbonyl, etc.

(3) for the guanidino group of Arg, protection may be by mitro, tosyl, CBZ, adamantyloxycarbonyl, 2,2,5,7,8-pentamethylchroman-6-sulfonyl or 2,3,6-trimethyl-4-methoxyphenylsulfonyl, or BOC.

(4) for the hydroxyl group of Ser, Thr, or Tyr, protection may be, for example, by C1-C4 alkyl, such as t-butyl; benzyl (BAL); substituted BZL, such as p-methoxybenzyl, p-nitrobenzyl, p-chlorobenzyl, o-chlorobenzyl, and 2,6-dichlorobenzyl.

(5) for the carboxyl group of Asp or Glu, protection may be, for example, by esterification using groups such as BZL, t-butyl, cyclohexyl, cyclopentyl, and the like.

(6) for the imidazole nitrogen of His, the tosyl moiety is suitable employed.

(7) for the phenolic hydroxyl group of Tyr, a protecting group such as tetrahydropyranyl, tert-butyl, trityl, BZL, chlorobenzyl, 4-bromobenzyl, or 2,6-dichlorobenzyl is suitably employed. The preferred protecting group is 2,6-dichlorobenzyl.

(8) for the side chain amino group of Asn or Gln, xanthyl (Xan) is preferably employed.

(9) for Met, the amino acid is preferably left unprotected.

(10) for the thio group of Cys, p-methoxybenzyl is typically employed.

The C-terminal amino acid, eg, Lys, is protected at the N-amino position by an appropriately selected protecting group, in the case of Lys, BOC. The BOC-Lys-OH can be first coupled to the benzyhydrylamine or chloromethylated resin according to the procedure set forth in Horiki et al, (Chemistry Letters, 165-168 1978) or using isopropylcarbodiimide at about 25° C. for 2 hours with stirring. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups are described in the literature.

After removal of the α-amino protecting group, the remaining α-amino and side-chain protected amino acids are coupled stepwise within the desired order. As an alternative to adding each amino acid separately in the synthesis, some may be coupled to one another prior to addition to the solid-phase synthesizer. The selection of an appropriate coupling reagent is within the skill of the art. Particularly suitable as a coupling reagent is N,N¹-dicyclohexyl carbodiimide or diisopropylcarbodiimide.

Each protected amino acid or amino acid sequence is introduced into the solid-phase reactor in excess, and the coupling is suitably carried out in a medium of dimethylformamide (DMF) or CH₂Cl₂ or mixtures thereof. If incomplete coupling occurs, the coupling procedure is repeated before removal of the N-amino protecting group piror to the coupling of the next amino acid. The success of the coupling reaction at each stage of the synthesis may be monitored. A preferred method of monitoring the synthesis is by the ninhydrin reaction, as described by Kaiser et al., Anal Biochem, 34: 595 (1970). The coupling reactions can be performed automatically using well known methods, for example, a BIOSEARCH 9500™ peptide synthesizer.

Upon completion of the desired peptide sequence, the protected peptide must be cleaved from the resin support, and all protecting groups must be removed. The cleavage reaction and removal of the protecting groups is suitably accomplished simultaneously or stepwise. When the resin support is a chloromethylated polystyrene resin, the bond anchoring the peptide to the resin is an ester linkage formed between the free carboxyl group of the C-terminal residue and one of the many chloromethyl groups present on the resin matrix. It will be appreciated that the anchoring bond can be cleaved by reagents that are known to be capable of breaking an ester linkage and of penetrating the resin matrix.

One especially convenient method is by treatment with liquid anhydrous hydrogen fluoride. This reagent not only will cleave the peptide from the resin but also will remove all protecting groups. Hence, use of this reagent will directly afford the fully deprotected peptide. When the chloromethylated resin is used, hydrogen fluoride treatment results in the formation of the free peptide acids. When the benzhydrylamine resin is used, hydrogen fluoride treatment results directly in the free peptide amines. Reaction with hydrogen fluoride in the presence of anisole and dimethylsulfide at 0° C. for one hour will simultaneously remove the side-chain protecting groups and release the peptide from the resin.

When it is desired to cleave the peptide without removing protecting groups, the protected peptide-resin can undergo methanolysis to yield the protected peptide-resin can undergo methanolysis to yield the protected peptide in which the C-terminal carboxyl group is methylated. The methyl ester is then hydrolysed under mild alkaline conditions to give the free C-terminal carboxyl group. The protecting groups on the peptide chain then are removed by treatment with a strong acid, such as liquid hydrogen fluoride. A particularly useful technique for methanolysis is that of Moore et al, Peptides, Proc Fifth Amer Pept Symp, M Goodman and J Meienhofer, Eds, (John Wiley, N.Y., 1977), p. 518-521, in which the protected peptide-resin is treated with methanol and potassium cyanide in the presence of crown ether.

Another method of cleaving the protected peptide form the resin when the chloromethylated resin is employed is by ammonolysis or by treatment with hydrazine. If desired, the resulting C-terminal amide or hydrazide can be hydrolysed to the free C-terminal carboxyl moiety, and the protecting groups can be removed conventionally.

It will also be recognized that the protecting group present on the N-terminal α-amino group may be removed preferentially either before or after the protected peptide is cleaved from the support.

If in the peptides being created carbon atoms bonded to four non identical substituents are asymmetric, then the compounds may exist as disastereoisomers, enantiomers or mixtures thereof. The syntheses described above may employ racemates, enantiomers or disastereoisomers as starting materials or intermediates. Disastereomeric products resulting from such syntheses may be separated by chromatographic or crystallization methods. Likewise, enantiomeric product mixtures may be separated using the same techniques or by other methods known in the art. Each of the asymmetric carbon atoms, when present, may be in one of two configurations (R or S) and both are within the scope of the present invention.

Purification of the peptide is typically achieved using conventional procedures such as preparative HPLC (including reversed phase HPLC) or other known chromatographic techniques such as gel permeation, ion exchange, partition chromatography, affinity chromatography (including monoclonal antibody columns) or counter-current distribution.

As described above, the peptide may be prepared as salts of various inorganic and organic acids and bases. A number of methods are useful for the preparation of these salts and are known to those skilled in the art. Examples include reaction of the free acid or free base form of the peptide with one or more molar equivalents of the desired acid or base in a solvent or solvent mixture in which the salt is insoluble; or in a solvent like water after which the solvent is removed by evaporation, distillation or freeze drying. Alternatively, the free acid or base form of the produce may be passed over an ion-exchange resin to form the desired salt or one salt form of the product may be convened to another using the same general process.

The starting materials required for use in the chemical synthesis of peptides described above are known in the literature or can be prepared using known methods and known starting materials.

In certain embodiments, the composition includes an agonist or an antagonist of a neuregulin receptor and especially at least one of HER-2, HER-3 and HER-4 receptors. Typically, these compounds affect the phosphorylation of these receptors.

Examples of agonists and antagonists and their manufacture are discussed in the following US patent applications and patents, the contents of which are incorporated by reference in their entirety:

| | |
|---|---|
| US20040138417A1 | Chimeric heteromultimeric adhesins functioning as antagonists or agonists |
| | of neuralgin - used to develop products for treating e.g. immunological, |
| | cardiac and neurological disorders, inflammatory disease and cancer |
| U.S. Pat. No. 6,696,290 | Chimeric heteromultimeric adhesins functioning as antagonists or agonists |
| | of neuralgin - used to develop products for treating e.g. immunological, |
| | cardiac and neurological disorders, inflammatory disease and cancer |
| US20030199020A1 | Chimeric heteromultimeric adhesins functioning as antagonists or agonists |
| | of neuralgin - used to develop products for treating e.g. immunological, |
| | cardiac and neurological disorders, inflammatory disease and cancer |
| US20020002276A1 | Novel chimeric heteromultimeric adhesin which comprises extracellular |
| | domain of natural heteromultimeric receptor and multimerization domain, |
| | and binds to ligand of receptor, useful for inhibiting receptor activation |

Other examples of agonists or antagonists include antibodies that affect the binding of neuregulin to its receptor.

In certain embodiments, the composition for inducing tissue regeneration further includes a neurotrophin, examples of which are nerve growth factor (NGF), brain derived neurotrophic factor (BDNF), neurotrophin-3 (NT-3), neurotrophin-4 (NT-4) and neurotrophin-5 (NT-5) and Ciliary Neurotrophic Factor (CNTF). These are described further herein.

In other embodiments the composition for inducing tissue regeneration further includes more than one neurotrophin or growth factor.

In particular embodiments the composition includes neuropeptide Y (NPY).

The composition may also further include a fibroblast growth factor, such as fibroblast growth factor 1 (FGF-1), fibroblast growth factor 2 (FGF-2), fibroblast growth factor 3 (FGF-3), fibroblast growth factor 4 (FGF-4), fibroblast growth factor 8 (FGF-8), fibroblast growth factor 9 (FGF-9), fibroblast growth factor 10 (FGF-10), fibroblast growth factor 17a (FGF-17a).

In another embodiment there is provided a method for inducing regeneration of a mammalian tissue at a site of injury in the tissue. The method includes the steps of providing an agent selected from the group consisting of: a neuregulin; a fragment of a neuregulin; an agonist or antagonist of a receptor for neuregulin; to a site of injury in a mammalian tissue to induce regeneration of the tissue at the site of injury.

Typically the agent is provided to the site of injury by contacting the tissue with neuregulin, one or more fragments thereof, a neuregulin variant or an agonist or antagonist of a neuregulin receptor.

It will be understood that in certain embodiments, neuregulin and compositions containing neuregulin may be provided to the site of injury by contacting the tissue with a molecule that induces expression of a neuregulin. In other embodiments, a neuregulin fragment is provided to the site of injury by contacting the tissue with an enzyme for converting a neuregulin isoform to a functional fragment. In still further embodiments, the neuregulin is provided to the site of injury by contacting the tissue with an agent for preventing the degradation of neuregulin.

In other embodiments, the method includes applying a composition as described above to the site of injury. In these embodiments, the composition typically includes a neurotrophin, and especially NGF.

Further, in certain embodiments, neuregulin is provided to the site of injury by providing a cell that expresses or otherwise produces neuregulin to the site of injury. Examples of such cells include stem cells, progenitor cells and precursor cells. These cells include cells that express p75 as described further below. Other examples of cells include neuregulin cell transfectants that express neuregulin.

Thus in another embodiment there is provided a method for inducing regeneration of a mammalian tissue at a site of injury in the tissue, the method including the step of providing a cell that expresses neuregulin or a fragment thereof to a site of injury in a mammalian tissue to induce regeneration of the tissue at the site of injury.

Typically the tissue is human tissue, although it will be understood that the agent may be useful for inducing regeneration in other mammalian tissues. Further, the neuregulin, one or more fragments thereof is typically selected to correspond to the species from which the neuregulin or fragment thereof may be obtained.

Typically the tissue to be regenerated is selected from the group consisting of skin, muscle, such as cardiac muscule, fat, bone, or any tissue derived from the group of endoderm, mesoderm, ectoderm or combination thereof and including bone, cartilage, muscle, connective tissue, tendon, nerve, adipose, skin, gastrointestinal tissue, heart, organs, cornea, optical tissue, exocrine and/or endocrine glands. For example if subcutaneous fatty tissue were to be regenerated it would include include the regeneration of the primary cell type i.e. fat, and its blood supply (vascular tissue) nerve supply and stromal tissue (supporting structures including ECM, basil lamina etc). Similarly this concept can be used to support the regeneration of most tissues e.g. for muscle it will be myocytes, vascular supply and nerve supply and stromal tissue.

In certain embodiments, nerve tissue is generated such that the primary cell type i.e. the neuron and its associated extensions such as dendrites, axons, terminal boutons, and any respective specialised sensory structures (e.g. corpuscles) are partially or wholly regenerated. Additionally regeneration may include supporting cells such as neuroglia or myelinated cells and supporting structures such as stromal tissue, supporting structures (including ECM, basil lamina etc) and blood supply (vascular tissue).

The tissue to be regenerated may be tissue injured, lost, or atrophied by disease processes or degeneration or aging. Such tissues could be the spinal cord (for example, multiple sclerosis), the substantia nigra in Parkinson's disease, or the olfactory mucosa or Alzheimer's disease. It will be understood that Neuregulin and/or a Neurotrophin may be provided in individuals predisposed to multiple sclerosis, Parkinson's or Alzheimer's disease, or to individuals having symptoms of onset of these diseases for preventing or reducing the severity of these diseases.

It will be understood that the method is particular useful for the regeneration of tissue at a site of injury is selected from the group consisting of laceration, burns, surgical incisions and excisions/removals/debridements, abrasion, puncture, amputation, excision.

In certain embodiments, the invention is useful for the therapy of individuals requiring regeneration of spinal cord tissue. Thus the tissue may be spinal cord or neural tissue such as brain, spinal cord, peripheral nerves, optic nerves, retina, cranial nerves and autonomic nerves.

Where the site of injury is an external surface, in particular, skin, in certain embodiments the agent may be administered topically. Other forms of administration, such as oral, nasal, subcutaneous or intravenous administration may be selected in accordance with the location of the particular tissue injury. For example, ischaemic pancreatic tissue may best be treated by oral administration of the agent.

In one embodiment, the neuregulin, variant or fragment thereof is provided in an amount of between about 1 to 500 ng per mm³ of tissue to be regenerated. In certain embodiments, amounts within this range may be useful, for example from about 2 to 250 ng per mm³ of tissue to be regenerated, 5 to 100 ng per mm³ of tissue to be regenerated, and 10 to 50 ng per mm³ of tissue to be regenerated. In most circumstances, about 10 ng of neuregulin per mm³ of tissue to be regenerated is a suitable amount for most tissue types. Appropriate amounts of neuregulin for tissue regeneration in given circumstances can be determined by one skilled in the art according to the disclosures herein.

In certain embodiments, and especially where tissue injury is to be treated by topical application, useful amounts of neuregulin would be from about 1 to 100 ng per mm² of tissue to be regenerated, although other amounts could be used, for example from about 5 to 75 ng per mm² of tissue to be regenerated, from about 7.5 to 50 ng per mm² of tissue to be regenerated and from about 10 to 25 ng per mm² of tissue to be regenerated.

It will be understood that the agent may be co-administered with other compounds, including compounds that facilitate or induce angiogenesis, neurogenesis, anti-inflammatory compounds and antibiotic compounds. Fibroblast growth factors have been described above. Other compounds include PDGF, IGF-1, TGF-beta, EGF, SPARC and the like.

It will be understood that the agent may be co-administered with either somatic stem cells and/or other cells having plasticity, such as olfactory stem cells or with agents that induce plasticity in cells, or agents that promote the recruitment of stem cells to the site of injury e.g. GMSCF, SCF.

In certain embodiments the cells express p75, otherwise known as CD271 or the low affinity nerve growth factor receptor (LNGFR). Examples of these cells are olfactory ensheathing cells (OECs), neural stem cells, neural progenitor cells, neurospheres, mesenchymal stem cells, oesophageal keratinocyte stem cells and certain sub-populations of fibroblasts.

It will be understood that in certain embodiments, the plastic cells may be p75 negative-i.e. they are cells that have little or no p75 molecules on the cell surface.

In certain embodiments the cells are plastic fibroblasts. Examples of these cells may be fibroblasts that have been induced to express p75, or other fibroblasts such as, those expressing REX-1, OCT3/4 and others markers as have been described (see Rieske, Krynska and Azizi; Differentiation (2005) 73:474-483).

It will be understood that the agent will typically be applied in the form of a medicament, such as a composition, formulation or dressing. Accordingly, in certain embodiments, there is provided a use of an agent selected from the group consisting of neuregulin, a fragment of neuregulin; a compound for inducing expression of the neuregulin gene; and an agonist or antagonist of a neuregulin receptor in the manufacture of a medicament for inducing regeneration of a mammalian tissue at a site of injury in the tissue. The neuregulin peptide, fragment thereof, compound forinducing expression of neuregulin and agonist or antagonist of the neuregulin receptor useful in this embodiment of the invention are as described above.

In certain embodiments the medicament further includes other compounds, including compounds that facilitate or induce angiogenesis, neurogenesis, anti-inflammatory compounds and antibiotic compounds, examples of which are identified above.

The medicament further comprises a pharmaceutically carrier, excipient, diluent or lubricant.

Typically, where the site of injury is in skin or subcutaneous tissue, the medicament is to be provided in a form for topical application, such as a liquid, for example a cream or lotion, a semi-solid state, such as a gel or a solid state, such as a powder. A composition for the sustained release neuregulin, is particularly useful.

In other embodiments, the medicament is provided on, or in the form of a dressing such as a bandage, gauze pad, adhesive plaster or other like surgical or therapeutic dressing.

Accordingly, there is provided a dressing for inducing regeneration of a mammalian tissue at a site of injury in the tissue, the dressing including an agent selected from the group consisting of neuregulin, a fragment of neuregulin, a compound for inducing expression of the neuregulin, and an agonist or antagonist of a neuregulin receptor.

Processes for the adsorption of a peptide or other compound onto a dressing are known to one skilled in the art.

The neuregulin peptide, a fragment thereof, a compound for inducing expression of neuregulin and an agonist or antagonist of the neuregulin receptor useful in this embodiment are as described above.

In certain embodiments the medicament further includes other compounds, including compounds that facilitate or induce angiogenesis, neurogenesis, anti-inflammatory compounds and antibiotic compounds, examples of which are identified above.

In further embodiments there is provided a composition for inducing regeneration of a mammalian tissue at a site of injury in the tissue. The composition includes one or more of: a neurotrophin such as nerve growth factor (NGF) and neurotrophic factor 3 (NT-3); a fragment of a neurotrophin; a compound for inducing expression of a neurotrophin gene; and an agonist or antagonist of a neurotrophin receptor.

Other examples of a neurotrophin for use in the composition include BDNF, NT-4, NT-5 and Ciliary Neurotrophic Factor (CNTF). Typically the neurotrophin is NGF or NT-3.

The NGF or NT-3 for use in the composition is typically syngeneic. In other words, if the mammalian tissue is human tissue, the NGF is typically human NGF and the NT-3 is typically human NT-3. However, in some circumstances improved tissue regeneration is observed with molecules from other species.

In other circumstances, certain fragments of a NGF or NT-3, such as a naturally occurring isoform may provide improved tissue regeneration.

An example of NGF includes a peptide having an amino acid sequence shown in SwissProt accession number P01138.

It will be understood, however, that the NGF may have an amino acid sequence that is not the same as, but rather, has homology with the sequence referenced by SwissProt reference: P01138. These are described herein as a “NGF variant”. In some embodiments, an NGF variant has a sequence that is at least 70% homologous to the sequence referenced by SwissProt reference P01138 shown above. In other embodiments the NGF variant sequence is at least 75% homologous to the sequence referenced by SwissProt reference P01138 shown above. In other embodiments the NGF variant sequence is at least 80% homologous to the sequence referenced by SwissProt reference P01138 shown above. In other embodiments the NGF variant sequence is at least 85% homologous to the sequence referenced by SwissProt reference P01138 shown above. In other embodiments the NGF variant sequence is at least 90% homologous to the sequence referenced by SwissProt reference P01138 shown above. In other embodiments the NGF variant sequence is at least 95% homologous to the sequence referenced by SwissProt reference P01138 shown above. In other embodiments the NGF variant sequence is at least 98% homologous to the sequence shown in SEQ ID No: 1. In other embodiments the NGF variant sequence is at least 99% homologous to the sequence referenced by SwissProt reference P01138 shown above.

It will be understood that in determining homology, regard is not to be had to peptide regions that are not included in a peptide that has been subjected to post translational modifications, such as, for example a leader or signal peptide.

Neutrophins such as nerve growth factor (NGF) and neurotrophic factor 3 (NT-3) can be produced by the processes discussed above in relation to the production of neuregulins.

An example of NT-3 includes a peptide having an amino acid sequence shown in SwissProt accession number: P20783.

It will be understood, however, that the NT-3 may have an amino acid sequence that is not the same as, but rather, has homology with the sequence referenced by SwissProt reference: P20783. These are described herein as a “NT-3 variant”. In some embodiments, an NT-3 variant has a sequence that is at least 70% homologous to the sequence referenced by SwissProt reference P20783 shown above. In other embodiments the NT-3 variant sequence is at least 75% homologous to the sequence referenced by SwissProt reference P20783 shown above. In other embodiments the NT-3 variant sequence is at least 80% homologous to the sequence, referenced by SwissProt reference P20783 shown above. In other embodiments the NT-3 variant sequence is at least 85% homologous to the sequence referenced by SwissProt reference P20783 shown above. In other embodiments the NT-3 variant sequence is at least 90% homologous to the sequence referenced by SwissProt reference P20783shown above. In other embodiments the NT-3 variant sequence is at least 95% homologous to the sequence referenced by SwissProt reference P20783 shown above. In other embodiments the NT-3 variant sequence is at least 98% homologous to the sequence shown in SEQ ID No: 1. In other embodiments the NT-3 variant sequence is at least 99% homologous to the sequence referenced by SwissProt reference P20783 shown above.

It will be understood that in determining homology, regard is not to be had to peptide regions that are not included in a peptide that has been subjected to post translational modifications, such as, for example a leader or signal peptide.

In certain embodiments, the composition may include an agonist or antagonist of a NGF receptor or a NT-3 receptor. Typically these compounds have activity against the low affinity nerve growth factor receptor (LNGFR) although other receptors such as tyrosine kinase receptors, especially trkA and trkC are contemplated.

Examples of agonists and antagonists and their manufacture are discussed in the following US patent applications and patents, the contents of which are incorporated by reference in their entirety:

| | | |
|---|---|---|
| U.S. Pat. No. 5,958,875 | New nerve growth factor agonist peptide(s) - used for | The Regents of |
| | treating disorders involving NGF responsive cells, e.g. | the University |
| | Alzheimer's disease, neuropathies, neural injury or | of California |
| | apoptosis | |
| U.S. Pat. No. 6,291,247 | Inhibition of neurotrophin activity - using factor that | Queen's |
| | interferes with sub-unit interaction | University at |
| | | Kingston |
| U.S. Pat. No. 5,134,121 | New nerve growth factor peptide(s) - with agonist and/or | Regents of the |
| | antagonist activity | University of |
| | | California |
| US20050265994A1 | Treating bone cancer pain in individual, involves | — |
| | administering nerve growth factor antagonist to individual | |
| US20040228862A1 | Use of nerve growth factor antagonist for treatment of post- | SHELTON |
| | surgical pain e.g. resting, mechanically induced pain, | DAVID L. |
| | thermally-induced pain, allodynia and hyperalgesia | |
| U.S. Pat. No. 5,990,129 | Use of 2-aryl-3-aroylbenzo(b)thiophene derivatives such as | Eli Lilly and |
| | raloxifene for up-regulating expression of trkA | Company |
| US20040253244A1 | Treating pain e.g. post-surgical pain comprises | SHELTON |
| | administering anti-nerve growth factor antibody in | DAVID L. |
| | combination with non-steroidal antiinflammatory drug | |
| US20040237124A1 | New anti-nerve growth factor antibodies for preventing or | PONS JAUME |
| | treating pain, including post-surgical pain, rheumatoid | |
| | arthritis pain or osteoarthritis pain, or for treating | |
| | inflammatory cachexia associated with rheumatoid arthritis | |
| US20040170651A1 | Delivery of a composition to the central nervous system or | ROUX SYLVIE |
| | spinal cord - comprises administration of a non-toxic, | |
| | proteolytic fragment of tetanus toxin in association with a | |
| | molecule having biological function | |
| US20030104995A1 | Ameliorating neuronal degeneration such as Huntington's | REILLY |
| | Chorea, Alzheimer's or Parkinson's disease in a subject, by | JENNIFER OTT |
| | administering to the subject a composition comprising | |
| | hedgehog therapeutic and neurotrophic factor | |
| US20030049244A1 | Method of stimulating oocyte maturation to alleviate | SEIFER DAVID B. |
| | infertility comprises administration of brain derived | |
| | neurotrophic factor, neurotrophin-4/5 or nerve growth | |
| | factor | |
| U.S. Pat. No. 6,029,114 | Identifying optimal molecular structures by variable basis | Queen's |
| | Monte Carlo method - particularly to determine biologically | University at |
| | active conformation(s) of neurotrophin domains involved in | Kingston |
| | receptor binding and subsequent evolution of active ligands | |
| US20040071701A1 | Use of a nerve growth factor antagonist for the manufacture | DELAFOY |
| | of a medicament for the prevention or treatment of chronic | LAURE |
| | visceral pain | |

Other examples of agonists or antagonists include antibodies that affect the binding of NGF to its receptor, or the binding of NT-3 to its receptor.

In certain embodiments the composition may include an agent for modulating the amount of P75 expressed by a cell.

In certain embodiments, the composition for inducing tissue regeneration further includes a neuregulin, an example of which is heregulin. Other examples include isoforms of neuregulin.

In other embodiments the composition for inducing tissue regeneration further includes more than one neurotrophin or growth factor.

The composition may also further include a fibroblast growth factor, such as fibroblast growth factor 1 (FGF-1), fibroblast growth factor 2 (FGF-2), fibroblast growth factor 3 (FGF-3), fibroblast growth factor 4 (FGF-4), fibroblast growth factor 8 (FGF-8), fibroblast growth factor 9 (FGF-9), fibroblast growth factor 10 (FGF-10), fibroblast growth factor 17a (FGF-17a).

In particular embodiments the composition includes neuropeptide Y (NPY).

In another embodiment there is provided a method for inducing regeneration of a mammalian tissue at a site of injury in the tissue. The method includes the steps of providing an agent selected from the group consisting of: a neurotrophin such as nerve growth factor (NGF) and neurotrophic factor 3 (NT-3); a fragment of a neurotrophin; an agonist or antagonist of a receptor for a neurotrophin; to a site of injury in a mammalian tissue to induce regeneration of the tissue at the site of injury.

Typically the agent is provided to the site of injury by contacting the tissue with neurotrophin, one or more fragments thereof, a neurotrophin variant or an agonist or antagonist of a neurotrophin receptor.

It will be understood that in certain embodiments, neurotrophin may be provided to the site of injury by contacting the tissue with a molecule that induces expression of a neurotrophin. In other embodiments, a neurotrophin fragment is provided to the site of injury by contacting the tissue with an enzyme for converting a neurotrophin isoform to a functional fragment. In still further embodiments, the neurotrophin is provided to the site of injury by contacting the tissue with an agent for preventing the degradation of neurotrophin.

In other embodiments, the method includes applying a composition as described above to the site of injury. In these embodiments, the composition typically includes a neuregulin.

Further, in certain embodiments, neurotrophin is provided to the site of injury by providing a cell that expresses or otherwise produces neurotrophin to the site of injury. Examples of such cells include stem cells, progenitor cells and precursor cells. These cells may express p75 as described further below. Other examples of cells include neurotrophin cell transfectants that express neurotrophin.

Thus in another embodiment there is provided a method for inducing regeneration of a mammalian tissue at a site of injury in the tissue, the method including the step of providing a cell that expresses neurotrophin or a fragment thereof to a site of injury in a mammalian tissue to induce regeneration of the tissue at the site of injury.

Typically the tissue is human tissue, although it will be understood that the agent may be useful for inducing regeneration in other mammalian tissues. Further, the neurotrophin, one or more fragments thereof is typically selected to correspond to the species from which the neurotrophin or fragment thereof may be obtained.

Typically the tissue to be regenerated is selected from the group consisting of skin, muscle such as cardiac muscle, fat, bone, or any tissue derived from the group of endoderm, mesoderm, ectoderm or combination thereof and including bone, cartilage, muscle, connective tissue, tendon, nerve, adipose, skin, gastrointestinal tissue, heart, organs, cornea, optical tissue, exocrine and/or endocrine glands. For example if subcutaneous fatty tissue were to be regenerated it would include the regeneration of the primary cell type i.e. fat, and its blood supply (vascular tissue) nerve supply and stromal tissue (supporting structures including ECM, basil lamina etc). Similarly this concept can be used to support the regeneration of most tissues e.g. for muscle it will be myocytes, vascular supply and nerve supply and stromal tissue.

In certain embodiments, nerve tissue is generated such that the primary cell type i.e. the neuron and its associated extensions such as dendrites, axons, terminal boutons, and any respective specialised sensory structures (e.g. corpuscles) are partially or wholly regenerated. Additionally regeneration may include supporting cells such as neuroglia or myelinated cells and supporting structures such as stromal tissue supporting structures (including ECM, basil lamina etc) and blood supply (vascular tissue).

It will be understood that the method is particular useful for the regeneration of tissue at a site of injury is selected from the group consisting of laceration, burns, surgical incisions and excisions/removals/debridements, abrasion, puncture, amputation, excision.

The tissue to be regenerated may be tissue injured, lost, or atrophied by disease processes or degeneration or aging. Such tissues could be the spinal cord (for example, multiple sclerosis), the substantia nigra in Parkinson's disease, or the olfactory mucosa or Alzheimer's disease. It will be understood that Neuregulin and/or a Neurotrophin may be provided in individuals predisposed to multiple sclerosis, Parkinson's or Alzheimer's disease, or to individuals having symptoms of onset of these diseases for preventing or reducing the severity of these diseases.

The method of the invention is particular useful for the therapy of individuals having a deficiency in wound healing. An example of individual is a diabetic. Thus in a particularly preferred embodiment, the tissue is diabetic tissue.

In certain embodiments, the invention is useful for the therapy of individuals requiring regeneration of spinal cord tissue. Thus the tissue may be spinal cord or neural tissue such as brain, spinal cord, peripheral nerves, optic nerves, retina, cranial nerves and autonomic nerves.

Where the site of injury is an external surface, in particular, skin, in certain embodiments the agent may be administered topically. Other forms of administration, such as oral, nasal, subcutaneous or intravenous administration may be selected in accordance with the location of the particular tissue injury. For example, ischaemic pancreatic tissue may best be treated by oral administration of the agent.

In one embodiment, the neurotrophin, variant or fragment thereof is provided in an amount of between about 1 to 500 ng per mm³ of tissue to be regenerated. In certain embodiments, amounts within this range may be useful, for example from about 2 to 250 ng per mm³ of tissue to be regenerated, 5 to 100 ng per mm³ of tissue to be regenerated, and 10 to 50 ng per mm³ of tissue to be regenerated. In most circumstances, about 50-100 ng ng of NT-3 per mm³ of tissue to be regenerated is a suitable amount for most tissue types. About 100-200 ng ng of NGF per mm³ of tissue to be regenerated is a suitable amount for most tissue types. Appropriate amounts of neurotrophin for tissue regeneration in given circumstances can be determined by one skilled in the art according to the disclosures herein.

In certain embodiments, and especially where tissue injury is to be treated by topical application, useful amounts of neurotrophin would be from about 1 to 100 ng per mm² of tissue to be regenerated, although other amounts could be used, for example from about 5 to 75 ng per mm² of tissue to be regenerated, from about 7.5 to 50 ng per mm² of tissue to be regenerated and from about 10 to 25 ng per mm² of tissue to be regenerated.

It will be understood that the agent may be co-administered with other compounds, including compounds that facilitate or induce angiogenesis, neurogenesis, anti-inflammatory compounds and antibiotic compounds. Fibroblast growth factors have been described above. Other compounds include PDGF, IGF-1, TGF-beta, EGF, SPARC and the like.

It will be understood that the agent may be co-administered with either somatic stem cells and/or other cells having plasticity, or with agents that induce plasticity in cells, or agents that promote the recruitment of stem cells to the site of injury e.g. GMSCF, SCF.

In certain embodiments the cells express p75, otherwise known as CD271 or the low affinity nerve growth factor receptor (LNGFR). Examples of these cells are olfactory ensheathing cells (OECs), olfactory stem cells, neural stem cells, neural progenitor cells, neurospheres, mesenchymal stem cells, oesophageal keratinocyte stem cells and certain sub-populations of fibroblasts.

It will be understood that in certain embodiments, the plastic cells may be p75 negative-i.e. they are cells that have little or no p75 molecules on the cell surface.

In certain embodiments the cells are plastic fibroblasts. Examples of these cells are fibroblasts that have been induced to express p75.

It will be understood that the agent will typically be applied in the form of a medicament, such as a composition, formulation or dressing. Accordingly, in certain embodiments, there is provided a use of an agent selected from the group consisting of neurotrophin such as nerve growth factor (NGF) and neurotrophic factor 3 (NT-3), a fragment of neurotrophin; a compound for inducing expression of the neurotrophin gene; and an agonist or antagonist of a neurotrophin receptor in the manufacture of a medicament for inducing regeneration of a mammalian tissue at a site of injury in the tissue. The neurotrophin peptide, fragment thereof, compound for inducing expression of neurotrophin and agonist or antagonist of the neurotrophin receptor useful in this embodiment of the invention are as described above.

In certain embodiments the medicament further includes other compounds, including compounds that facilitate or induce angiogenesis, neurogenesis, anti-inflammatory compounds and antibiotic compounds, examples of which are identified above.

The medicament further comprises a pharmaceutically carrier, excipient, diluent or lubricant.

Typically, where the site of injury is in skin or subcutaneous tissue, the medicament is to be provided in a form for topical application, such as a liquid, for example a cream or lotion, a semi-solid states, such as a gel or a solid state, such as a powder. A composition for the sustained release neurotrophin is particularly useful.

In other embodiments, the medicament is provided on, or in the form of a dressing such as a bandage, gauze pad, adhesive plaster or other like surgical or therapeutic dressing.

Accordingly, there is provided a dressing for inducing regeneration of a mammalian tissue at a site of injury in the tissue, the dressing including an agent selected from the group consisting of neurotrophin such as nerve growth factor (NGF) and neurotrophic factor 3 (NT-3), a fragment of neurotrophin, a compound for inducing expression of the neurotrophin, and an agonist or antagonist of a neurotrophin receptor.

Processes for the adsorption of a peptide or other compound onto a dressing are known to one skilled in the art.

The neurotrophin peptide, a fragment thereof, a compound for inducing expression of neurotrophin and an agonist or antagonist of the neurotrophin receptor useful in this embodiment are as described above.

In certain embodiments the medicament further includes other compounds, including compounds that facilitate or induce angiogenesis, neurogenesis, anti-inflammatory compounds and antibiotic compounds, examples of which are identified above.

### Examples

### Example 1

### Protein Identification by Mass Spectrometry

Peptide digest was separated by online cation exchange (SCX) and C₁₈ nano-LC using an Ultimate HPLC, Switchos and Famos auto sampler system (LC-Packings, Amsterdam, Netherlands). CM (50 mL) was TCA precipitated and the resulting pellet (˜20 μg) was digested with 5 μg trypsin in 50 μL, 20 mM NH₄HCO₃ for 16 hr at 37° C. Peptides (10 μL) were diluted in 100 μL of 0.1% (v/v) formic acid and loaded onto a SCX micro trap (1×8 mm, Michrom Bioresources, Auburn, Calif.) at 20 μL/min. Peptides were eluted using 20 μL volumes of ammonium acetate in a stepwise gradient range of 5-1000 mM. The unbound load fraction and each salt step were concentrated and desalted using a micro C₁₈ pre-column (500 μm×2 mm, Michrom Bioresources) with H₂O:CH₃CN (98:2, 0.1% formic acid) at 20 μL/min. Following a 10 min wash the pre-column was switched (Switchos) into line with a fritless analytical C₁₈ column (75 μm×˜12 cm) and peptides eluted using a linear gradient of H₂O:CH₃CN (95:5, 0.1% formic acid-buffer A) to H₂O:CH₃CN (40:60, 0.1% formic acid-buffer B) at 200 nL/min over 30 min, High voltage (2300 V) was applied at the beginning of the column through a low volume tee (Upchurch Scientific, Oak Harbor, Wash.) and the outlet positioned ˜1 cm from the orifice of an API Qstar Pulsar I hybrid tandem mass spectrometer (Applied Biosystems). Positive ions were generated by electrospray and the Qstar operated in information dependent acquisition mode. An MS survey scan was acquired (m/z 350-1700, 0.75 s) and the 2 largest multiply charged ions (counts>20, charge state≧2 and ≦4) sequentially selected by Q1 for MS-MS analysis. Tandem mass spectra were accumulated for 2 seconds (m/z 65-2000). Processing scripts generated data suitable for submission to the database search program MASCOT. Tolerance allowed was one missed cleavage, mass tolerance of ±0.2 Da; modifications included oxidation to methionines. Database searches were conducted according and accepted according to parameters outlined above. Moreover, those peptides sequenced with a statistically significant score as outlined by the MASCOT search engine were accepted.

Results

Match to: NRG1_XENLA Score: 61
(093383) Pro-neuregulin-1, membrane-bound isoform precursor (Pro-NRG1) [contains:
Neuregulin-1]
Nominal mass (Mᵣ): 75748; Calculated pl value: 9.37
NCBI BLAST search of NRG1 XENLA against nr
Unformatted sequence string for pasting into other applications
Taxonomy: Xenopus laevis
Variable modifications: Oxidation (M)
Cleavage by Trypsin: cuts C-term side of KR unless next residue is P
Sequence Coverage: 35%

Matched peptides shown in Bold Red

| | |
|---|---|
| 1 | MAEKKKVKEG KGRKGKGKKD RKGKKAEGSD QGAAASPKLK EIKTQSVQEG |
| 51 | KKLVLKCQAV SEQPSLKFRW FKGEKEIGAK NKPDSKPEHI KIRGKKKSSE |
| 101 | LQISKASSAD NGEYKCMVSN QLGNDTVTVN VTIVPKPTYN HLLLMKIYLK |
| 151 | VTSVEKSVEP STLNLLESQK EVIFATTKRG DTTAGPGHLI KCSDKEKTYC |
| 201 | VNGGECYVLN GITSSNQFMC KCKPGFTGAR CTETDPLRVV RSEKHLGIEF |
| 251 | MEAEELYQKR VLTITGICID LLVVGDMCVV DAYCKTKKQR KKLNDRLRQS |
| 301 | LRERNKNITN KDNRPHNPKN PPPRKNVQLV NQYVSKNVIS SEHVIERETE |
| 351 | TSFSTSHYTS TTHHSTTVTQ TPSHSWSNGL SESMISEKSY SVIVTSSVEN |
| 401 | SRHTSPTGPR GRLNGIGGPR DCSYLRHARD TPDSYRDSPH SERYVSAMTT |
| 451 | PARMSPVEFK TPISPKSPCL ETSPPESSLA VSVPSVAVSP FIEEERPLLL |
| 501 | VSPPRLREKR YDRKTPQKTP HKQHNSYHHN PGHDSSSLPP NPLRIVEDEE |
| 551 | YETTQEYEPS LEPAKKLVNS RRQKRTKPNG HISNRLELDS DSSSESSTSE |
| 601 | SETEDERIGE ETPFLSIQNP LAASLESASL YRHADSRTNP TSRFSTQEEL |
| 651 | QARLSSIANQ ALCDQKKRKM TCKTLFI |

| | | | | | | |
|---|---|---|---|---|---|---|
| Start-End | Observed | Mr(expt) | Mr(calc) | Delta | Miss | Sequence |

| | | | | | | |
|---|---|---|---|---|---|---|
| 25-38 | 658.83 | 1315.64 | 1315.64 | −0.00 | 1 | K.KAEGSDQGAAASPK.L |
| | | | | | | (Ions score 15) |
| 97-115 | 681.41 | 2041.21 | 2041.00 | 0.22 | 2 | K.KSSELQISKASSADNGEYK.C |
| | | | | | | (Ions score 1) |
| 147-156 | 590.40 | 1178.78 | 1178.69 | 0.09 | 1 | K.IYLKVTSVEK.S |
| | | | | | | (Ions score 1) |
| 157-179 | 864.01 | 2589.02 | 2589.40 | −0.38 | 2 | K.SVEPSTLNLLESQKEVIFATTKR.G |
| | | | | | | (Ions score 2) |
| 157-179 | 864.03 | 2589.06 | 2589.40 | −0.34 | 2 | K.SVEPSTLNLLESQKEVIFATTKR.G |
| | | | | | | (Ions score 2) |
| 157-179 | 864.03 | 2589.06 | 2589.40 | −0.34 | 2 | K.SVEPSTLNLLESQKEVIFATTKR.G |
| | | | | | | (Ions score 2) |
| 157-179 | 864.04 | 2589.10 | 2589.40 | −0.30 | 2 | K.SVEPSTLNLLESQKEVIFATTKR.G |
| | | | | | | (Ions score 3) |
| 171-178 | 454.77 | 907.52 | 907.50 | 0.02 | 0 | K.EVIFATTK.R |
| | | | | | | (Ions score 2) |
| 171-178 | 454.77 | 907.52 | 907.50 | 0.02 | 0 | K.EVIFATTK.R |
| | | | | | | (Ions score 11) |
| 171-178 | 454.77 | 907.52 | 907.50 | 0.02 | 0 | K.EVIFATTK.R |
| | | | | | | (Ions score 6) |
| 171-178 | 454.77 | 907.53 | 907.50 | 0.03 | 0 | K.EVIFATTK.R |
| | | | | | | (Ions score 2) |
| 171-191 | 737.90 | 2210.68 | 2211.20 | −0.52 | 2 | K.EVIFATTKRGDTTAGPGHLIK.C |
| | | | | | | (Ions score 2) |
| 222-241 | 736.00 | 2204.98 | 2205.11 | −0.14 | 2 | K.CKPGFTGARCTETDPLRVVR.S |
| | | | | | | (Ions score 2) |
| 325-336 | 710.34 | 1418.67 | 1418.79 | −0.12 | 1 | R.KNVQLVNQYVSK.N |
| | | | | | | (Ions score 2) |
| 389-402 | 509.90 | 1526.69 | 1526.76 | −0.07 | 0 | K.SYSVIVTSSVENSR.H |
| | | | | | | (Ions score 4) |
| 454-466 | 738.90 | 1475.79 | 1475.77 | 0.02 | 1 | R.MSPVEFKTPISPK.S Oxidation (M) |
| | | | | | | (Ions score 1) |
| 467-507 | 877.96 | 4384.74 | 4384.34 | 0.41 | 1 | K.SPCLETSPPESSLAVSVPSVAVSPFIEEERPLLLVSPPRLR.E |
| | | | | | | (Ions score 3) |
| 506-510 | 351.20 | 700.38 | 700.43 | −0.05 | 2 | R.LREKR.Y |
| | | | | | | (Ions score 7) |
| 545-566 | 876.35 | 2626.02 | 2626.22 | −0.20 | 1 | R.IVEDEEYETTQEYEPSLEPAKK.L |
| | | | | | | (Ions score 1) |
| 545-566 | 876.40 | 2626.18 | 2626.22 | −0.04 | 1 | R.IVEDEEYETTQEYEPSLEPAKK.L |
| | | | | | | (Ions score 4) |
| 545-566 | 876.41 | 2626.20 | 2626.22 | −0.02 | 1 | R.IVEDEEYETTQEYEPSLEPAKK.L |
| | | | | | | (Ions score 2) |
| 545-571 | 799.86 | 3195.43 | 3195.55 | −0.12 | 2 | R.IVEDEEYETTQEYEPSLEPAKKLVNSR.R |
| | | | | | | (Ions score 2) |
| 545-571 | 799.87 | 3195.45 | 3195.55 | −0.10 | 2 | R.IVEDEEYETTQEYEPSLEPAKKLVNSR.R |
| | | | | | | (Ions score 1) |
| 608-632 | 902.72 | 2705.13 | 2705.39 | −0.26 | 0 | R.IGEETPFLSIQNPLAASLESASLYR.H |
| | | | | | | (Ions score 0) |
| 608-632 | 677.48 | 2705.88 | 2705.39 | 0.49 | 0 | R.IGEETPFLSIQNPLAASLESASLYR.H |
| | | | | | | (Ions score 1) |
| 669-677 | 542.79 | 1083.57 | 1083.58 | −0.01 | 2 | R.KMTCKTLFI.- |
| | | | | | | (Ions score 1) |

Match to: NT3_XENLA Score: 35
(P25435) Neurotrophin-3 precursor (NT-3) (Neurotrophic factor) (HDNF) (Nerve growth
factor 2)
(NGF-
Nominal mass (Mᵣ): 30003; Calculated pl value: 9.31
NCBI BLAST search of NT3 XENLAagainst nr
Unformatted sequence string for pasting into other applications
Taxonomy: Xenopus laevis
Variable modifications: Oxidation (M)
Cleavage by Trypsin: cuts C-term side of KR unless next residue is P
Sequence Coverage: 51%

Matched peptides shown in Bold Red

| | |
|---|---|
| 1 | MSILFYVMFL PYLCGIHATN MDKRNLPENS MNSLFIKLIQ ADLLKNKISK |
| 51 | QTVDTKENHQ STIPKPQILL DLDGDDNMKQ DFQPVISLEA ELVKQQKQRR |
| 101 | YKSPRVLLSD SLPLEPPPLY LMDDYIGHST VVNNRTSRRK RFAEHKGHRG |
| 151 | EYSVCDSESL WVTDKMNAID IRGHQVTVLG EIKTGNSPVK QYFYETRCKE |
| 201 | ARPVKNGCRG IDDKHWNSQC KTSQTYVRAL TSENNKMVGW RWIRIDTSCV |
| 251 | CALSRKIGRS |

| | | | | | | |
|---|---|---|---|---|---|---|
| Start-End | Observed | Mr(expt) | Mr(calc) | Delta | Miss | Sequence |

| | | | | | | |
|---|---|---|---|---|---|---|
| 1-24 | 720.54 | 2878.13 | 2878.40 | −0.27 | 1 | -.MSILFYVMFLPYLCGIHATNMDKR.N Oxidation (M) |
| | | | | | | (Ions score 3) |
| 1-24 | 720.54 | 2878.14 | 2878.40 | −0.26 | 1 | -.MSILFYVMFLPYLCGIHATNMDKR.N Oxidation (M) |
| | | | | | | (Ions score 1) |
| 1-24 | 576.64 | 2878.16 | 2878.40 | −0.25 | 1 | -.MSILFYVMFLPYLCGIHATNMDKR.N Oxidation (M) |
| | | | | | | (Ions score 1) |
| 25-37 | 753.96 | 1505.91 | 1505.75 | 0.16 | 0 | R.NLPENSMNSLFIK.L |
| | | | | | | (Ions score 0) |
| 38-47 | 578.37 | 1154.74 | 1154.70 | 0.03 | 1 | K.LIQADLLKNK.I |
| | | | | | | (Ions score 4) |
| 38-47 | 578.38 | 1154.74 | 1154.70 | 0.03 | 1 | K.LIQADLLKNK.I |
| | | | | | | (Ions score 6) |
| 38-47 | 678.38 | 1154.75 | 1154.70 | 0.04 | 1 | K.LIQADLLKNK.I |
| | | | | | | (Ions score 6) |
| 38-47 | 578.38 | 1154.75 | 1154.70 | 0.04 | 1 | K.LIQADLLKNK.I |
| | | | | | | (Ions score 4) |
| 38-47 | 578.38 | 1154.75 | 1154.70 | 0.05 | 1 | K.LIQADLLKNK.I |
| | | | | | | (Ions score 4) |
| 38-47 | 578.38 | 1154.75 | 1154.70 | 0.05 | 1 | K.LIQADLLKNK.I |
| | | | | | | (Ions score 4) |
| 38-47 | 578.38 | 1154.76 | 1154.70 | 0.05 | 1 | K.LIQADLLKNK.I |
| | | | | | | (Ions score 7) |
| 38-47 | 678.39 | 1154.76 | 1154.70 | 0.06 | 1 | K.LIQADLLKNK.I |
| | | | | | | (Ions score 4) |
| 38-47 | 578.39 | 1154.76 | 1154.70 | 0.06 | 1 | K.LIQADLLKNK.I |
| | | | | | | (Ions score 8) |
| 80-99 | 795.37 | 2383.10 | 2383.29 | −0.19 | 2 | K.QDFQPVISLEAELVKQQKQR.R |
| | | | | | | (Ions score 1) |
| 80-99 | 596.78 | 2383.10 | 2383.29 | −0.18 | 2 | K.QDFQPVISLEAELVKQQKQR.R |
| | | | | | | (Ions score 1) |
| 100-105 | 403.73 | 805.45 | 805.46 | −0.01 | 2 | R.RYKSPR.V |
| | | | | | | (Ions score 2) |
| 184-197 | 423.19 | 1688.74 | 1688.82 | −0.07 | 1 | K.TGNSPVKQYFYETR.C |
| | | | | | | (Ions score 1) |
| 200-214 | 829.41 | 1656.80 | 1656.84 | −0.04 | 2 | K.EARPVKNGCRGIDDK.H |
| | | | | | | (Ions score 2) |
| 229-236 | 438.75 | 875.48 | 875.43 | 0.05 | 0 | R.ALTSENNK.M |
| | | | | | | (Ions score 3) |
| 229-236 | 438.75 | 875.48 | 875.43 | 0.05 | 0 | R.ALTSENNK.M |
| | | | | | | (Ions score 8) |
| 229-236 | 438.75 | 875.49 | 875.43 | 0.06 | 0 | R.ALTSENNK.M |
| | | | | | | (Ions score 11) |
| 229-236 | 438.79 | 875.56 | 875.43 | 0.13 | 0 | R.ALTSENNK.M |
| | | | | | | (Ions score 1) |
| 229-236 | 438.79 | 875.67 | 875.43 | 0.13 | 0 | R.ALTSENNK.M |
| | | | | | | (Ions score 1) |
| 229-241 | 753.37 | 1504.72 | 1504.75 | −0.03 | 1 | R.ALTSENNKMVGWR.W |
| | | | | | | (Ions score 3) |
| 229-241 | 507.91 | 1520.71 | 1520.74 | −0.03 | 1 | R.ALTSENNKMVGWR.W Oxidation (M) |
| | | | | | | (Ions score 2) |
| 229-244 | 659.62 | 1975.84 | 1976.00 | −0.17 | 2 | R.ALTSENNKMVGWRWIR.I Oxidation (M) |
| | | | | | | (Ions score 3) |
| 229-244 | 659.64 | 1975.91 | 1976.00 | −0.10 | 2 | R.ALTSENNKMVGWRWIR.I Oxidation (M) |
| | | | | | | (Ions score 1) |
| 245-259 | 811.40 | 1620.79 | 1620.84 | −0.05 | 2 | R.IDTSCVCALSRKIGR.S |
| | | | | | | (Ions score 2) |

rms error 68 ppm
Match to: NT4_XENLA Score: 16
(P24727) Neurotrophin-4 precursor (NT-4)
Nominal mass (Mᵣ): 26197; Calculated pl value: 8.84
NCBI BLAST search of NT4 XENLA against nr
Unformatted sequence string for pasting into other applications
Taxonomy: Xenopus laevis
Variable modifications: Oxidation (M)
Cleavage by Trypsin: cuts C-term side of KR unless next residue is P
Sequence Coverage: 38%

Matched peptides shown in Bold Red

| | |
|---|---|
| 1 | MILRLYAMVI SYCCAICAAP FQSRTTDLDY GPDKTSEASD RQSVPNNFSH |
| 51 | VLQNGFFPDL SSTYSSMAGK DWNLYSPRVT LSSEEPSGPP LLFLSEETVV |
| 101 | HPEPANKTSR LKRASGSDSV SLSRRGELSV CDSVNVWVTD KRTAVDDRGK |
| 151 | IVTVMSEIQT LTGPLKQYFF ETKCNPSGST TRGCRGVDKK QWISECKAKQ |
| 201 | SYVRALTIDA NKLVGWRWIR IDTACVCTLL SRTGRT |

| | | | | | | |
|---|---|---|---|---|---|---|
| Start-End | Observed | Mr(expt) | Mr(calc) | Delta | Miss | Sequence |

| | | | | | | |
|---|---|---|---|---|---|---|
| 79-112 | 738.58 | 3687.87 | 3687.94 | −0.06 | 2 | R.VTLSSEEPSGPPLLFLSEETVVHPEPANKTSRLK.R |
| | | | | | | (Ions score 3) |
| 143-166 | 858.04 | 2571.10 | 2571.39 | −0.30 | 2 | R.TAVDDRGKIVTVMSEIQTLTGPLK.Q |
| | | | | | | (Ions score 5) |
| 149-166 | 966.05 | 1930.08 | 1930.08 | 0.00 | 1 | R.GKIVTVMSEIQTLTGPLK.Q Oxidation (M) |
| | | | | | | (Ions score 1) |
| 149-166 | 644.37 | 1930.09 | 1930.08 | 0.00 | 1 | R.GKIVTVMSEIQTLTGPLK.Q Oxidation (M) |
| | | | | | | (Ions score 1) |
| 151-173 | 538.65 | 2688.20 | 2688.41 | −0.21 | 1 | K.IVTVMSEIQTLTGPLKQYFFETK.C Oxidation (M) |
| | | | | | | (Ions score 3) |
| 151-182 | 716.15 | 3575.71 | 3575.80 | −0.09 | 2 | K.IVTVMSEIQTLTGPLKQYFFETKCNPSGSTTR.G |
| | | | | | | (Ions score 3) |
| 205-220 | 637.96 | 1910.86 | 1911.08 | −0.22 | 2 | R.ALTIDANKLVGWRWIR.I |
| | | | | | | (Ions score 2) |
| 205-220 | 637.96 | 1910.87 | 1911.08 | −0.21 | 2 | R.ALTIDANKLVGWRWIR.I |
| | | | | | | (Ions score 1) |
| 205-220 | 637.98 | 1910.93 | 1911.08 | −0.15 | 2 | R.ALTIDANKLVGWRWIR.I |
| | | | | | | (Ions score 2) |

rms error 76 ppm

### Example 2

### Inducing Regeneration of Mammalian Tissue at a Site of Tissue Injury with NT3

32 male Wistar rats were anaesthetized by an intramuscular injection of Ketamine and Xylaxine. The dorsum was shaved and disinfected. Two full thickness skin wounds of 1 cm (square shaped) were made.

In each rat, in one wound a single slow release pellet of NT3 containing either 0.5 μg, 5 μg, 10 μg, or 50 μg was placed. In the other wound a control pellet (not containing NT3) was placed. 8 rats were assigned to each dose treatment.

Animals were allowed to recover and wound size was assessed every 3 days for 15 days. The animals were sacrificed at 6 weeks, and wounds were both grossly and histologically examined.

Both wounds were excised en bloc and placed into fixative (10% buffered formalin). Multiple cross sections were cut across the wound area perpendicular to epidermis to include epidermis, dermis and subcutaneous tissue.

Sections were processed, embedded in paraffin and cut into 5 μm thick sections. The sections were then stained with Haematoxylin and Eosin and Masson's Trichrome.

Tissue regeneration was then evaluated by assessment of the following parameters:

A. Wound length was measured with an optical micrometer as the distance between intact epidermis. Re-epithelisation was calculated by the measuring new epithelium as a percentage of wound length.

B. Collagen deposition was assessed in dermis in the centre of the wound and graded semi quantitatively as 0 (no collagen deposition); 1+ (slight collagen deposition); 2+ (moderate collagen deposition); 3+ (heavy collagen deposition).

C. Degree of angiogenesis was assessed by number of capillary sprouts in dermis.

D. Degree of inflammation was assessed by evaluating the number of neutrophils, lymphocytes, macrophages, plasma cells, eosinophils and foreign body type of giant cells semi quantitatively on a scale of 0 to 3 (0-absent, 1-mild, 2-moderate and 3-severe).

E. Any necrosis (or absence thereof) was noted.

F. Cell proliferation is assessed by special stains ki67 and PCNA.

G. Regeneration of the epidermal appendages was noted by their reinstallation .

H. Overall regeneration was scored as 1—poor regeneration; 2—moderate regeneration; 3—good regeneration of epidermis and dermis; 4—regeneration of epidermis, dermis and evidence of re-installation of the epidermal appendages

### Example 3

### Inducing Regeneration of Porcine Tissue at a Site of Injury with NT4

Four healthy, male domestic pigs weighing approximately 20-30 kg were sedated with Ketamine 20 mg/kg IM+Xylazine, 2.0 mg/kg IM.

An endotracheal tube was placed while maintaining spontaneous ventilation and anaesthesia maintained using isoflurane (0.5%-2.0%) administered in a mixture of appropriate oxygen/nitrous oxide.

Each animal underwent four aseptic dorsal skin incisions. In brief local hair was shaved to clear way for four 2 cm full thickness skin incisions. Each incision was made down to a depth of 10 mm. A standard sterile number 22 blade (Swann-Morton) was used. One incision was allocated per quadrant of dorsal skin as not to interfere with the other incisions. All incisions were produced with a single stroke to a premarked measured line. All wounds were closed with a subcutaneous layer of 3-0 braided absorbable Vicryl suture (Ethicon Ltd, UK).

NT4 was dissolved in serial dilutions of 3 mg/mL pH neutral collagen gel giving final concentrations of 0.1 μg/mL, 1 μg/mL, and 10 μg/mL concentrations of NT4. An additional gel with no NT4 was used as a control. The same solution was applied topically daily to the same wound edge for 5 days under Tegaderm (3M) transparent adhesive dressings to localize the NT4-gel to the site. Within each pig there was an incision exposed to each concentration of of NT4.

Buprenorphine, 0.075 mg/kg IMI was administered immediately after surgery for analgesia with additional doses provided as required.

Wound sites were observed and evaluated subjectively for wound edge approximation, progression of regeneration, evidence of inflammation or infection, and incisional dehiscence daily for the first 5 days after surgery and on a regular basis thereafter. At 28 days animals were euthanized and wound sites were harvested en bloc as a 3×6-cm squares and placed in 10% buffered formalin histopathologic examination.

Histopathologic specimens for evaluation pf regeneration and scoring were fixed in 10% buffered formalin for a minimum of 48 hours. Cross sections of each wound were processed using standard methods, embedded in paraffin, cut at 5 μm, and mounted on glass slides. Individual sections for each wound were stained and were examined under a light microscope for assessment as based on Example 2.

### Example 4

### Inducing Regeneration of Mammalian Tissue at a Site of Tissue Injury with a Nucleic Acid Encoding for a Neurotrophin to the Site of Injury

In this example, BDNF is provided to a wound site for tissue regeneration according to Example 2. However, BDNF is provided to the wound site by providing replication-defective adenoviral vectors [Adv.Rous sarcoma virus (RSV)-nf] encoding for brain-derived neurotrophic factor (BDNF) and these were expanded and injected directly into the injury site. An example of this expression method is shown in Baumgartner, B J, and Shine, H D, 1997; J. Neurosci. 17, 6504-6511.

1. A method for inducing regeneration of a mammalian tissue at a site of injury in the tissue including providing a neuregulin and/or a neurotrophin to a site of injury in a mammalian tissue to induce regeneration of the tissue at the site of injury.

2. A method for inducing regeneration of a mammalian tissue at a site of injury in the tissue including providing a fragment of a neuregulin and/or a neurotrophin to a site of injury in a mammalian tissue to induce regeneration of the tissue at the site of injury.

3. A method for inducing regeneration of a mammalian tissue at a site of injury in the tissue including providing an agonist or antagonist of a neuregulin receptor and/or a neurotrophin receptor to a site of injury in a mammalian tissue to induce regeneration of the tissue at the site of injury.

4. A method according to claim 1 wherein the neuregulin and/or neurotrophin is provided to the site of injury in the tissue by providing a cell that produces a neuregulin and/or neurotrophin at the site of injury in the tissue.

5. A method according to claim 1 wherein the neuregulin is provided to the site of injury in the tissue by providing a nucleic acid encoding neuregulin and/or a neurotrophin to the site of injury in the tissue.

6. A method according to claim 1 further including providing an agent selected from the group consisting of FGF, PDGF, TGF, IGF, EGF, GMSCF and SCF to the site of injury in the tissue.

7. A method according to claim 1 further including providing neuropeptide Y (NPY) to the site of injury.

8. A method according to claim 1 further including providing a morphogen such as a retinoic acid to the site of injury in the tissue.

9. A method according to claim 1 further including providing a wnt protein to the site of injury in the tissue.

10. A method according to claim 1 further including providing a stem cell, progenitor cell, or a precursor cell that is capable of differentiating to regenerate the elements of the tissue.

11. A method according to claim 10 wherein the cell is an epithelial stem cell, olfactory stem cell, neural stem cell, neural progenitor cell, neurosphere, or mesenchymal stem cell.

12. A method according to claim 1 further including providing an olfactory ensheathing cell.

13. A method according to claim 1 wherein the neuregulin and/or neurotrophin is provided to the site of injury in the tissue on a dressing.

14. A method according to claim 1 wherein the neuregulin and/or neurotrophin is provided to the site of injury in the tissue on a scaffold.

15. A method according to claim 1 wherein the tissue is neural tissue or dermal tissue.
